(19) United States
(12) Patent Application Publication
(10) Pub. No.: US 2004/0142855 A1
(43) Pub. Date: Jul. 22, 2004

(54) Stem cell engraftment-enhancing cellular proteins and their uses

(75) Inventors: Yolonda L. Colson, Needham, MA (US); Matthew J. Schuchert, Pittsburgh, PA (US)

Correspondence Address: JONES DAY, 222 EAST 41ST ST, NEW YORK, NY, 10017, US

(73) Assignee: University of Pittsburgh

(21) Appl. No.: 10/375,376

(22) Filed: Feb. 27, 2003

Related U.S. Application Data
(63) Continuation of application No. 09/692,060, filed on Oct. 19, 2000, now abandoned.
(60) Provisional application No. 60/161,108, filed on Oct. 22, 1999.

(51) Int. Cl. A61K 38/17; C07K 14/47

(52) U.S. Cl. 514/8; 530/395

(57) ABSTRACT

The present invention relates to the isolation and identification of cellular proteins and protein complexes that promote the engraftment of allogeneic stem cells and the induction of immunologic tolerance in recipient transplant hosts. More specifically, the present invention relates to a novel 33 kD glycoprotein, p33, that can form a complex with the T cell receptor (TCR) β chain, alone or in association with the CD3 antigen. The presence of the p33 protein, the TCRβ/p33 complex or the CD3/TCRβ/p33 complex of the invention on the surface of cells correlates with the ability of those cells to facilitate allogeneic engraftment in vivo. The compositions and methods of this invention are useful for promoting the engraftment of allogeneic cells and tissues in vivo, for the reduction of Graft Versus Host Disease (GVHD) which occurs in connection with transplantation of allogeneic cells in vivo and for the induction of immunologic tolerance to donor cells and tissue in vivo, e.g., in solid organ or tissue transplantation or in bone marrow transplantation used in connection with the treatment of leukemia or other hematological diseases.

The present application claims the benefit under 35 U.S.C. 119(e) of co-pending provisional application Serial No. 60/161,108, filed on Oct. 22, 1999, which is incorporated herein by reference in its entirety.

### 1. INTRODUCTION

The present invention relates to the isolation and identification of cellular proteins and protein complexes that promote the engraftment of allogeneic stem cells and the induction of immunologic tolerance in recipient transplant hosts. More specifically, the present invention relates to a novel 33 kD glycoprotein, p33, that can form a complex with the T cell receptor (TCR) β chain, alone or in association with the CD3 antigen. The presence of the p33 protein, the TCRβ/p33 complex or the CD3/TCRβ/p33 complex of the invention on the surface of cells correlates with the ability of those cells to facilitate allogeneic engraftment in vivo. The compositions and methods of this invention are useful for promoting the engraftment of allogeneic cells and tissues in vivo, for the reduction of Graft Versus Host Disease (GVHD) which occurs in connection with transplantation of allogeneic cells in vivo and for the induction of immunologic tolerance to donor cells and tissue in vivo, e.g., in solid organ or tissue transplantation or in bone marrow transplantation used in connection with the treatment of leukemia or other hematological diseases.

### 2. BACKGROUND OF THE INVENTION

A major goal in solid organ transplantation is the engraftment of the donor organ without a graft rejection immune response generated by the recipient, while preserving the immunocompetence of the recipient against other foreign antigens. Typically, nonspecific immunosuppressive agents such as cyclosporine, methotrexate, steroids and FK506 are used to prevent host rejection responses. They must be administered on a daily basis and if stopped, graft rejection usually results. However, nonspecific immunosuppressive agents function by suppressing all aspects of the immune response, thereby greatly increasing a recipient's susceptibility to infections and diseases, including cancer.

Furthermore, despite the use of immunosuppressive agents, graft rejection still remains a major source of morbidity and mortality in human organ transplantation. Only 50% of heart transplants survive 5 years and 20% of kidney transplants survive 10 years (see Powles et al., Feb. 16, 1980, Lancet 1 (8164): 327-329; Ramsay et al., 1982, New Engl. J. Med. 306(No. 7): 392-397). Most human transplants fail within 10 years without permanent acceptance. It would therefore be a major advance if immunological tolerance can be induced in the recipient.

The only known clinical condition in which complete systemic donor-specific transplantation tolerance occurs reliably and reproducibly is when chimerism is created through bone marrow transplantation (see Qin et al., 1989, J. Exp. Med. 169: 779; Sykes et al., 1988, Immunol. Today 9: 23; Sharabi et al., 1989, J. Exp. Med. 169: 493). This has been achieved in neonatal and adult animal models as well as in humans by total lymphoid irradiation of a recipient followed by bone marrow transplantation with donor cells. The widespread application of bone marrow transplantation to areas outside of malignancy has been limited by GVHD. GVHD results from the ability of immunocompetent mature immune cells (mainly T cells, but some B cells and natural killer cells) in the donor graft to recognize host tissue antigens as foreign and to invoke an adverse immunologic reaction. Thus, the success rate of bone marrow transplantation is, in part, dependent on the ability to closely match the major histocompatibility complex (MHC) of the donor cells with that of the recipient cells. The MHC is a gene complex that encodes a large array of individually unique glycoproteins expressed on the surface of both donor and host cells that are the major targets of transplantation rejection immune responses. In the human, the MHC is referred to as HLA. When HLA identity is achieved by matching a patient with a family member such as a sibling, the probability of a successful outcome is relatively high, although GVHD is still not completely eliminated. The incidence and severity of GVHD are directly correlated with degree of genetic disparity. In fact, only one or two antigen mismatches are acceptable because GVHD is very severe in cases of greater disparities. When allogeneic bone marrow transplantation is performed between two MHC-mismatched individuals of the same species, common complications involve failure of engraftment, poor immunocompetence and a high incidence of GVHD.

GVHD is a potentially lethal complication in bone marrow transplantation, which occurs in about 35-50% of recipients of untreated HLA-identical marrow grafts (Martin et al., 1985, Blood 66: 664) and up to 80% of recipients of HLA-mismatched marrow. Unfortunately, only 30% of patients generally have a suitably matched HLA-identical family member donor, and thus most patients are either excluded from being considered for bone marrow transplantation, or if they are transplanted must tolerate a high risk of GVHD. Although mixed allogeneic reconstitution, in which a mixture of donor and recipient marrow is transplanted, results in improved immunocompetence and increased resistance to GVHD, successful engraftment is still not consistently achieved and GVHD still often occurs.

Recent studies in bone marrow transplantation suggest that the major cause of GVHD are T cells, as the removal of T cells from the donor cell preparation was associated with a reduction in the incidence of GVHD (see, e.g., Vallera et al., 1989, Transplant. 47: 751; Rayfield et al., 1984, Eur. J. Immunol. 14(No. 4): 308-313; Vallera et al., 1982, J. Immunol. 128: 871; Korngold et al., 1978, J. Exp. Med. 148(No. 6): 1687-1689; Prentice et al., Mar. 3, 1984, Lancet 1(8375): 472-476). After T cells were implicated to be the predominant mediator of GVHD in animal models, aggressive protocols for T-cell depletion (TCD) of human donor bone marrow were instituted. Although the incidence of GVHD was decreased dramatically, TCD was accompanied by a significant increase in the failure of engraftment, indicating that T cells might also play a positive role in bone marrow engraftment (see, e.g., Soderling et al., 1985, J. Immunol. 135: 941; Vallera et al., 1982, Transplant. 33: 243; Pierce et al., 1989, Transplant. 48(No. 2): 289-296). The increase in failure of engraftment in human recipients ranged from about 5-70% of total patients and was related to the degree of MHC disparity between the donor and recipient (Blazar et al., 1987, UCLA Symp. on Molecular Cellular Biology, New Series, 4^{th} International Symposium, Keystone, Colo., April 13-18, 1986, Vol. 53: 381-398; Filipovich et al., 1987, Transplant. 44(No. 1): 62-69; Martin et al., 1985, Blood 66: 664; Martin et al., 1988, Adv. Immunol. 40: 379). Patients with failed engraftment usually die, even if a second bone marrow transplant is performed. Consequently, most transplant institutions in the United States have abandoned TCD of donor bone marrow and, thus, must tolerate a high level of GVHD which leads to significant morbidity and mortality. Thus, the application of bone marrow transplantation as a form of treatment is limited only to settings where the potential of GVHD is clearly outweighed by the potential benefit of transplantation. It was anticipated that the administration of purified bone marrow stem cells would optimize engraftment and avoid GVHD. However, recent studies have shown that purified bone marrow stem cells only engraft in genetically identical, but not in genetically disparate recipients.

The implication that T cells might participate in both harmful GVHD reactions and helpful engraftment facilitation was an enigma that existed for a long time in the scientific community. Investigators began to search for the possible existence of a bone marrow component which could facilitate bone marrow engraftment but was removed during TCD. Identification and purification of this facilitating component would potentially allow the design of transplant protocols to selectively prevent GVHD, while preserving the cells that enhance engraftment. Although most investigators speculated that the facilitating component was a hematopoietic cell distinct from the hematopoietic stem cells, such a component had never been identified or characterized until recently. In fact, all evidence pointed towards the involvement of some form of T cells.

However, it has recently been discovered that a unique bone marrow-derived cell population referred to as FC (Facilitatory or Facilitating Cells) facilitates engraftment of allogeneic hematopoietic stem cells across major and minor MHC disparities in a recipient without producing GVHD (Kaufman et al., 1994, Blood 84 (No. 8): 2436-2446). Thus, whereas the administration-of purified donor stem cells alone failed to achieve reliable engraftment across allogeneic barriers, the administration of purified allogeneic donor stem cells plus donor FC cells results in durable multilineage chimerism and donor-specific transplantation tolerance without GVHD (Kaufman et al., supra).

FC are isolated from normal bone marrow via multiparameter flow cytometric cell sorting and are identified by the phenotypic characteristic of CD8αβ and CD3ε expression on their cell surface, in the absence of conventional αβ and γβ-TCR heterodimers (i.e., CD3⁺, CD8⁺, αβTCR- and γδTCR−). In addition, the FC express several markers shared by other leukocytes. The isolation and identification of specific markers, e.g., proteins, expressed by FC would greatly assist the rapid isolation of this cell type, e.g., via the production of antibodies to the protein markers. Moreover, if proteins expressed by FC could be identified as correlating with the stem cell-engrafting and tolerance-inducing abilities of the FC, those proteins would provide new ways of enhancing allogeneic donor cell engraftment and donor-specific immunologic tolerance following transplantation.

Cell surface expression of CD3 without the conventional TCR proteins, as displayed by FC, has previously been described in developing thymocytes, but always in association with either TCRβ or a chaperone or surrogate protein such as calnexin (Ley et al., 1989, Eur. J. Immunol. 19: 2309; Groettrup et al., 1993, Eur. J. Immunol. 23: 1393-1396; Wiest et al., 1994, J. Exp. Med. 180: 1375-1382; and Wiest et al., 1995, EMBO J. 14 (No. 14): 3425-3433). Expression of such functional multisubunit CD3/TCR receptors is a tightly regulated process, requiring an organized sequence of CD3 and TCR gene expression (Snodgrass et al., 1985, Nature 313: 592-595 and von Bohmer, 1998, Ann. Rev. Immunol. 6: 309-326). Thus, successful CD3 surface expression relies upon coexpression of a classical αβ or γδ TCR heterodimer or alternative/additional chaperone or surrogate proteins, which promote receptor stability and prevent its degradation (Wiest et al., 1994, supra).

The CD3⁺/CD8⁺ phenotype expressed on the surface of FC without the usual αβ or γδ TCR heterodimer suggested to Applicants that perhaps an alternative CD3-associated protein is present on FC. A novel CD3-associated 33 kD protein and protein complex expressed on the surface of FC are disclosed in the present application.

### 3. SUMMARY OF THE INVENTION

The present invention relates to the isolation and identification of a novel 33 kD protein (referred to herein as “p33”), a novel TCRβ/p33 complex as well as a novel CD3/TCRβ/p33 complex, which protein and/or complexes are expressed on the surface of FC. The present invention is based, in part, on Applicants' discovery that the expression of the novel CD3/TCRβ/p33 protein complex of this invention directly correlates with the ability of FC to facilitate allogeneic engraftment of donor cells and tissues in vivo with the resultant induction of donor-specific tolerance. Thus, the proteins and protein complexes of this invention are useful for promoting allogeneic cell, tissue or organ engraftment and donor-specific tolerance in transplantation procedures in vivo, such as solid organ transplantation or bone marrow transplantation.

Other embodiments of this invention include biologically active fragments or derivatives of p33, recombinantly-produced p33 polypeptides, and the nucleic acid molecules, recombinant vectors and genetically-engineered host cells and organisms for the recombinant production of those p33 polypeptides. In addition, antibodies directed to the p33 proteins and polypeptides of the invention are also within the scope of this invention.

The present invention further includes methods for enhancing hematopoeitic stem cell engraftment in vivo, methods for inducing immunologic tolerance in vivo, and/or methods for reducing GVHD by administering to a patient in need thereof a therapeutically effective amount of the stem cell engraftment-enhancing protein (SEEP) p33, alone as the active pharmacologic agent, or in combination with TCRβ and/or CD3 as a complex. Alternatively, the p33 protein may be administered as a surface protein, alone or in complex with TCRβ and/or CD3, on naturally-occurring or genetically-engineered cells.

Thus, the pharmaceutical compositions of this invention include p33 protein compositions, TCRβ/p33 compositions, CD3/TCRβ/p33 compositions, cellular compositions comprising naturally-occurring cell populations having p33 or TCRβ/p33 or CD3/TCRβ/p33 on their surface or cellular compositions 10 comprising genetically-engineered cell populations having p33 or TCRβ/p33 or CD3/TCRβ/p33 on their surface.

### 4. BRIEF DESCRIPTION OF THE FIGURES

FIGS. 1A-1B. Flow cytometric analysis of FC and splenic T cells. FIG. 1A depicts the characteristic flow cytometric staining pattern of normal murine bone marrow utilizing CD8 mononclonal antibody 53-6.7, TCRβ monoclonal antibody H57-597, and γδTCR monoclonal antibody GL3. FC stained positive for CD8 but lacked the conventional TCR heterodimers seen in mature splenic T cells.

FIG. 1B is a histogram demonstrating that CD3 expression (as detected by monoclonal antibody 145-2c11) on FC differs significantly from mature splenic T cells.

FIGS. 2A-2C. Biotin Western blots of non-reduced (FIG. 2A) and reduced (FIG. 2B) anti-CD3ε immunoprecipitates (using monoclonal antibody 145-2c11) from lysates of sorted surface-biotinylated FC and T cells. FIG. 2C depicts a biotin Western blot of a reduced anti-TCRβ immunoprecipitate (using monoclonal antibody H57-597) from surface-biotinylated FC and T cell lysates. Each experiment utilized equal numbers of sorted cells (approximately 1×10⁵). Molecular weight markers are provided in kilodaltons.

FIG. 3. Biotin Western blots of two-dimensional non-reduced and reduced diagonal gels of anti-CD3e immunoprecipitates (using monoclonal antibody 145-2c11) from lysates of surface-biotinylated FC and T cells. The molecular masses of the reduced second PAGE (polyacrylamide gel electrophoresis) dimension are provided in kilodaltons on the left. The TCRαβ heterodimer on the T cell is seen as a doublet at 40-45 kD, whereas the FC-associated dimer is composed of molecules of 45 kD (TCRβ) and 33 kD (p33).

FIGS. 4A-4B. Biotin Western blots of serial immunoprecipitation experiments from lysates of surface-biotinylated FC, TCRα-KO (knock-out) thymocytes (Thy) and splenic T cells (T cell). FIG. 4A depicts immunoprecipitation using a TCRα monoclonal antibody (H28-710), followed by TCRβ (H57-597) immunoprecipitation of the remaining lysates, whereas FIG. 4B depicts an immuoprecipitation using anti-pTα antiserum as the initial immunoprecipitating antibody. These immunoprecipitations demonstrate that p33 is antigenically distinct from pTα on thymocytes and TCRα on T cells.

FIGS. 5A-5B. FIG. 5A is a Biotin Western blot from two-dimensional IEF (isoelectric focusing)-PAGE (polyacrylamide gel electrophoresis) analysis of FC and T cell CD3 (145-2c11) immunoprecipitates. FIG. 5B. depicts a Biotin Western blot of FC CD3 immunoprecipitate with and without PNGase F (peptidyl-N-glycosidase F) digestion.

FIGS. 6A-6C. Stem cell engraftment and p33 expression in TCRβ-deficient mice. FIG. 6A depicts the promotion of allogeneic stem cell engraftment when purified FC are added to the stem cell inoculum. FIG. 6B demonstrates that this effect is lost when CD8⁺/TCR- FC from TCRβ-KO (TCRβ^{-/-}) or RAG1-KO donor cells deficient in the ability to produce TCRβ are added to the stem cell inoculum. FIG. 6C is a biotin Western blot of a serial immunoprecipitation with anti-CD3 followed by anti-CD8 monoclonal antibody, depicting that, although CD8+ protein expression in CD8⁺/TCR- cell populations from TCRβ^{-/-} or RAG1^{-/-} mice is present, TCRβ-deficient mice were unable to express CD3, TCRβ or p33.

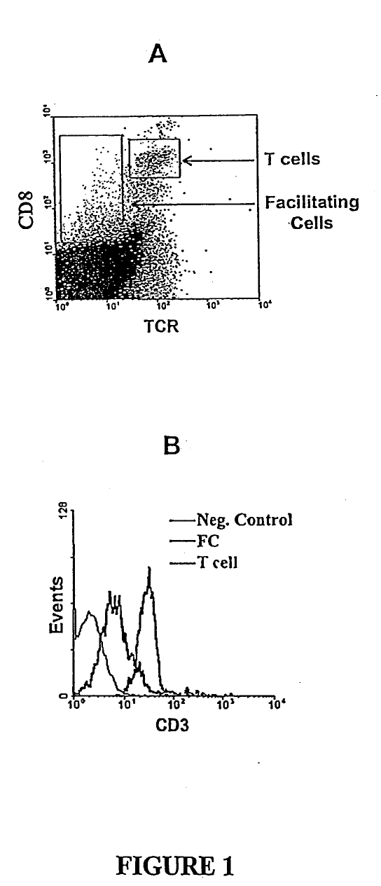

### 5. DETAILED DESCRIPTION OF THE INVENTION

The present invention relates to the isolation and identification of a novel stem cell engraftment-enhancing protein (“SEEP”). More specifically, the invention relates to a 33 kD protein, p33, alone or as part of a novel TCRβ/p33 or CD3/TCRβ/p33 protein complex, which protein and complexes are expressed on the surface of FC. The present invention is based in part on Applicants' discovery that the expression of the novel CD3/TCRβ/p33 protein complex of this invention directly correlates with the ability of FC to facilitate allogeneic engraftment of donor cells and tissues in vivo. Thus, the proteins and protein complexes of this invention are useful for promoting cell, tissue and organ engraftment and donor-specific tolerance in transplantation procedures, such as solid organ or bone marrow transplantation.

The novel p33 protein of this invention is typically found on the surface of the FC population of cells as part of a protein complex comprised of CD3, TCRβ and p33, with the p33 protein being in a disulfide linkage with the TCRβ chain. The p33 protein is further characterized herein as a glycoprotein with a rMW of 33 kD and an apparent isoelectric point of 4.5 (see Section 5.2 and Example Section 6, infra). Characterization of this protein infra reveals that it is different from any of the known CD3/TCRβ-associated proteins such as TCRα or or pre-Tα (“pTα”).

### 5.1. Isolation and Purification of the p33 Protein

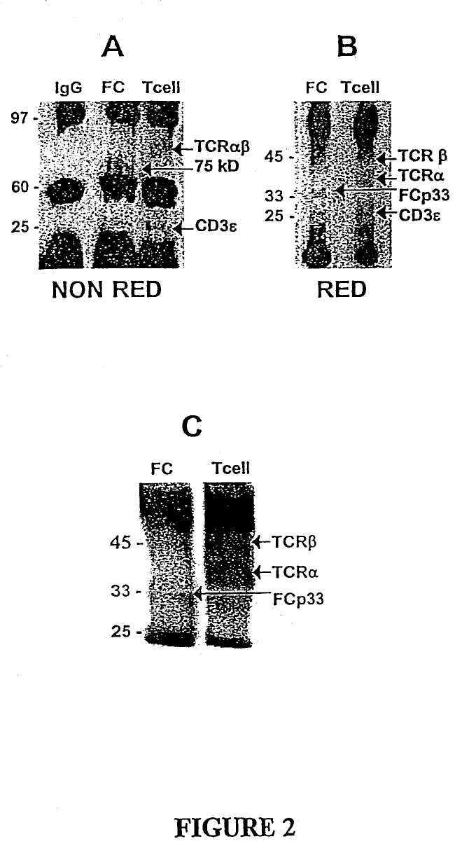

The p33 protein of this invention was isolated by immunoprecipitation and Western blotting of surface-biotinylated FC lysates with a monoclonal antibody to TCRβ and/or a monoclonal antibody to CD3ε. Under reducing conditions, using either antibody, identical 45 kD and 33 kD protein bands were obtained (see FIGS. 2B and 2C). This confirmed that the CD3-associated 45 kD protein on the FC surface is, in fact, TCRβ and that the novel 33 kD protein of the invention co-precipitates with both CD3 and TCRβ, suggesting that these three molecules are associated as a single complex on the FC surface.

More specifically, cell surface molecules from freshly-isolated FC and splenic T cells were labeled with Sulfo-N-Hydro succinimidester-LC-Biotin and solubilized in 1% Digitonin lysis buffer. CD3 and associated cell surface proteins were immunoprecipitated utilizing a monoclonal antibody specific for CD3ε (145-2C11, Pharmingen, San Diego Calif.) and visualized via SDS-PAGE and Western blotting (see Example Sections 6.2 and 6.3, infra).

As demonstrated in FIG. 2A, immunoprecipitation and Western blotting under nonreducing conditions revealed the presence of CD3 in both the FC and T cell lanes (25 kD). In addition, an 85 kD protein band was visualized in T cells consistent with the TCRα/TCRβ disulfide-linked heterodimer of the conventional TCR. In marked contrast, CD3 expression in the FC population was associated with a 75 kD band not observed in T cells. Subsequent analysis of the T cell lysates under reducing conditions revealed the expected 40 and 45 kD bands representing the TCRα and TCRβ proteins, respectively. The 75 kD complex seen in the FC sample was reduced to distinct protein bands of 45 and 33 kD (see FIG. 2B), suggesting that an alternative heterodimer is associated with CD3 in the FC population.

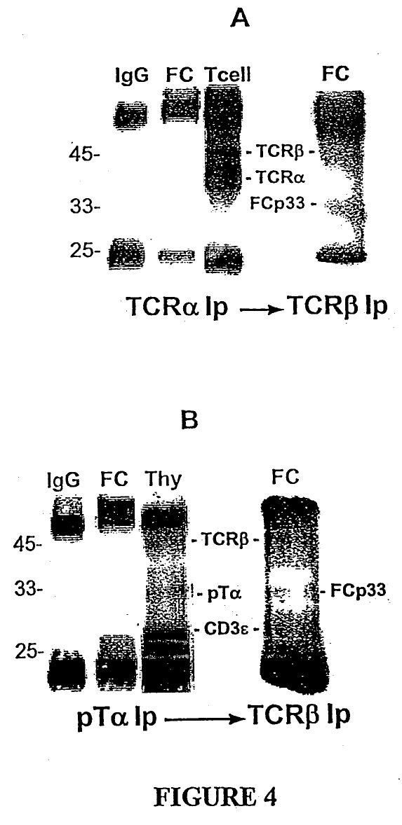

Next, immunoprecipitations and Western blotting utilizing a monoclonal antibody specific for TCRβ (H57-597) were performed on surface-biotinylated FC cell lysates. Under reducing conditions, TCRβ immunoprecipitation of FC lysates demonstrated the identical 45 and 33 kD protein bands detected in the CD3ε immunoprecipitation studies (FIG. 2C), confirming that the identity of the CD3-associated 45 kD protein on the FC surface is, in fact, TCRβ. Furthermore, the FC-associated 33 kD protein co-precipitated with both CD3 and TCRβ, suggesting that these three molecules are associated as a single complex on the FC surface. Finally, sequential immunoprecipitation studies with TCRα or pTα antibodies, followed by TCRβ antibody failed to reveal any TCRα or pTα proteins in the FC lysates, and hence, on the FC cell surface (see FIGS. 4A and 4B and Example Section 6.4, infra).

Thus, the p33 protein of this invention may be isolated via immunoprecipitation, followed by gel electrophoresis, either in the form of a 75 kD CD3/TCRβ/p33 protein complex (under non-reducing conditions) or as a single 33 kD protein (under reducing conditions). To isolate the CD3/TCRβ/p33 complex of the invention, the procedure used must be under non-reducing conditions that do not disrupt the bonding within the complex.

The isolation techniques employed to obtain the p33 protein of the invention involve a number of important parameters. First, because starting cell numbers (e.g., FC) and hence protein quantity are typically significantly limited, volumes were reduced in all steps of the isolation, e.g., washes, biotinylation, and immunoprecipitation phases, in order to minimize the loss of cells and protein. It was found that increasing the concentration of cells and protein enhanced the efficiency of the biotinylation and immunoprecipitation steps of the procedure.

In addition, it was important to use PBS as the initial washing reagent prior to the biotinylation procedure (as opposed to a standard TBS (Tris-Buffered Saline) reagent) because it was found that the amino group in buffers such as TBS can inactivate the biotin compound, thus preventing successful protein surface biotinylation. Post-immunoprecipitation washes preferably employed 0.1% Digitonin Wash Buffer×3, followed by 10 mM Tris buffer×2, which allowed for increased and more consistent p33 protein isolation.

In the biotinylation procedure utilized to isolate p33, a Sulfo-NHS-LC-Biotin reagent (Pierce, Rockford Ill.) was utilized as opposed to NHS-Biotin, a reagent that had been implicated in the art in altering the immunoreactivity of antigens (see, e.g., Kahne et al., 1994, J. Immunol. Methods 168: 209-218). The use of the Sulfo-NHS-LC-Biotin reagent resulted in reduced steric hindrance in the binding of the biotin to the secondary detection reagent, e.g., streptavidin-horseradish peroxidase conjugate, thus producing stronger signals with enhanced chemiluminescence.

The biotin concentration used was preferably in the range of 1-2 mg/ml since the reactive half-life of biotin is very limited and higher concentrations of the compound increase the probability that at least small amounts of protein will be detected. The biotinylation reaction is preferably carried out at room temperature.

The immunoprecipitation step of p33 isolation preferably utilizes an antibody concentration in the range of 2-5 mg/ml, which promotes antigen capture, and precipitation was preferably carried out using Protein G Sepharose (Pharmacia). Coupling of antibody to Sepharose prior to immunoprecipitation can limit loss of protein and non-specific noise. Finally, preclearance of non-specific proteins with non-specific antibody and Sepharose, a step known in the art to enhance the clarity of immunoprecipitation results, is disfavored in the isolation of p33 due to the fact that a substantial fraction of the p33 protein may be lost by this step in view of the small amounts of starting protein.

The p33 protein isolated as described above can be further purified by standard techniques known in the art, such as solubilization of the gel band which contains the protein and elution of the protein with an organic solvent or electroelution of the protein from the gel. More specifically, after separation of the p33 protein on the acrylamide gel, the protein should be extremely pure. It can be extracted from the gel by crushing the appropriate gel slice and eluting the protein utilizing an organic solvent, e.g., a mixture composed of formic acid/acetonitrile/isopropanol/H₂O (50/25/15/10 v/v/v/v) (see, e.g., Feick et al., 1990, Anal. Biochem. 187(2): 205-211). Higher sensitivity can be achieved by using SDS-free Laemelli (Tris-Glycine) electrophoresis buffer (see, e.g., Castellanos-Serra et al., 1996, Electrophoresis 17(10): 1564-1572). The protein can also be eluted from the gel by electroelution, using a variety of commercially available products, e.g., whole gel Eluter (BioRad, Hercules Calif., Catalog No. 165-1256). Once eluted, the p33 protein can be further analyzed for purity via HPLC and then sequenced.

### 5.2. Characterization of the p33 Protein and the CD3/TCRβ/p33 Complex

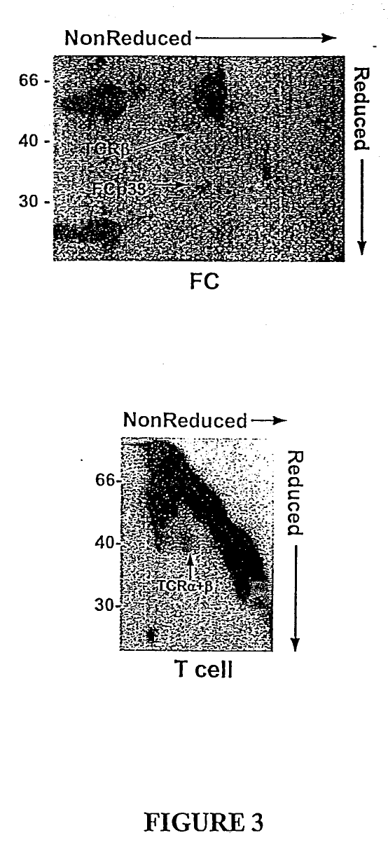

In order to further characterize the relationship of p33 to the CD3/TCR complex with which it is associated, nonreducing and reducing (“Diagonal”) two-dimensional electrophoresis was performed to detect the presence of disulfide bonds in the protein complex. More specifically, after immunoprecipitating surface-biotinylated proteins from FC and T cell lysates with CD3∈ monoclonal antibody, electrophoresis was sequentially performed under nonreducing and reducing conditions. The blots depicted in FIG. 3 demonstrate that FC possess a CD3-associated dimer of approximately 78 kD in the non-reduced dimension, that departs from the diagonal after reduction and separates into 45 kD (TCRβ) and 33 kD (p33) proteins positioned directly underneath. The expected 45 (TCRβ) and 40 (TCRα) kD products of the TCR heterodimer present in the T cell lysates are also shown for comparison. These data conclusively demonstrate that TCRβ and p33 exist as a disulfide-linked heterodimer, which is noncovalently associated with CD3 on the surface of the FC.

Taken together, the experiments described above and set forth in Example Section 6, infra, demonstrate that p33 is distinct from the TCRα, TCRβ and pTα proteins, and thus represents a unique 33 kD CD3-associated molecule which is expressed as part of a disulfide-linked TCRβ/p33 heterodimer on the surface of the FC.

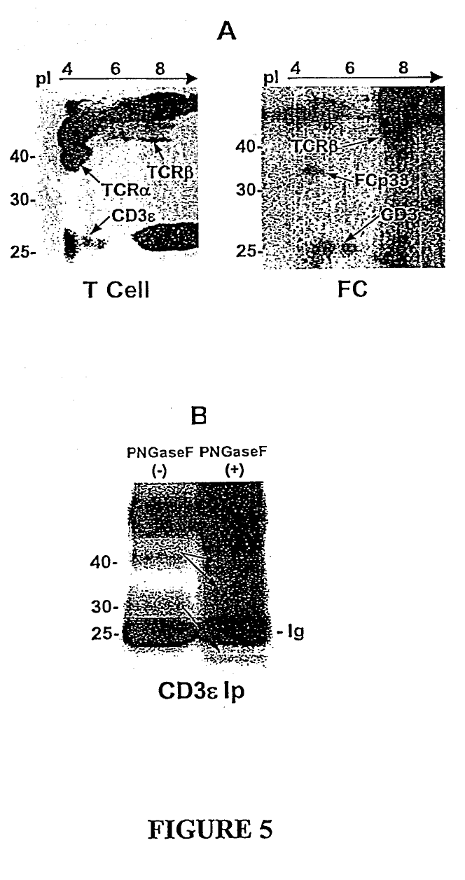

To further characterize the unique biochemical characteristics of p33, isoelectric focusing studies and glycosidase digests were performed. CD3 immunoprecipitates of FC lysates were first evaluated in isoelectric focusing studies. In addition to the expected CD3 (25 kD, Pi=5) and TCRβ (45 kD, Pi=8.5) bands, p33 was readily identified within the FC sample at a rMW of 33 kD and an apparent isoelectric point of 4.5 (see FIG. 5A and Example Section 6.5, infra). These characteristics distinguish p33 from all other reported CD3-associated proteins, as indicated in Table I below.

TABLE I

| | Mole- | | | | |
|---|---|---|---|---|---|
| | cular | Iso- | | | |
| | Weight | electric | N-Linked | Monoclonal | |
| Antigen | (kD) | Point | Residues | Antibody | Ref |
| TCRα | 40-44 | 4-4.5 | 4 | H28-710 | Becker (1989) |
| TCRβ | 42-45 | 8.2-8.7 | 4 | H57-597 | Kubo (1989) |
| TCRγ | 32-35 | 7.0-8.5 | 0-2 | HMT3.2 | Stingl (1987) |
| TCRδ | 45-46 | 5.9-7.9 | 3 | 3A10 | Chen (1996) |
| pTα | 33 | 6.8 | 2 | 7A1 | Kosugi (1997) |
| Calnexin | 90 | 4.0 | 0 | IP90 | Wiest (1995) |
| p33 | 33 | 4.5 | 2 | — | — |

Further, as is the case with other CD3/TCRβ-associated proteins, p33 is glycosylated, as evidenced by the reduction in molecular mass from 33 kD to 24 kD in the presence of the enzyme peptidyl-N-glycosidase F (see FIG. 5B). Taken together, these data further demonstrate that the p33 protein of this invention represents a biochemically distinct CD3/TCRβ-associated glycoprotein.

To date, three CD3/TCRβ cell surface complexes have been characterized: the classical TCR, where CD3/TCRβ is expressed in association with the TCRα chain (von Boehmer, 1998, Ann. Rev. Immunol. 6: 309-326), the pre-T cell receptor in which pTα is expressed in lieu of TCRα (Groettrup et al., 1993b, Cell 75: 283-294) and lastly, a CD3-associated TCRβ-β dimer complex that has been demonstrated in some transgenic systems (Groettrup et al., 1993a, Eur. J. Immunol. 23: 1393-1396). The individual chains of a TCRβ dimer migrate to identical 45 kD relative molecular weights (Groettrup et al., supra, 1993a and 1993b), such that a 90 kD complex on a non-reduced gel would be reduced to a single 45 kD species. The results from FIGS. 2 and 3 have already demonstrated that the p33 protein is distinct from TCRβ and is not consistent with a TCRβ-β dimer, as the 75-78 kD complex present on the FC surface in association with CD3 is reduced to 45 kD and 33 kD proteins. Since CD3ε or TCRβ immunoprecipitation of FC lysates results in the visualization of two protein species (45 kD and 33 kD), p33 could thus represent: a) a truncated TCRα protein with a resultant MW of 33 kD; b) the 33 kD pTα protein; or c) a unique 33 kD CD3/TCRβ-associated molecule.

Therefore, the presence of TCRα and pTα chains within the CD3/TCRβ/p33 complex was investigated using sequential immunoprecipitation studies (see FIGS. 4A and 4B and Example Section 6.5, infra). Biotinylated FC lysates were first immunoprecipitated with TCRα or pTα antibodies in order to remove any protein complexes that contained TCRα or pTα, respectively. The remaining supernatant was subsequently subjected to TCRβ immunoprecipitation in order to capture any remaining TCRβ complexes that did not contain TCRα or pTα proteins. The Western blots of these immunopreciptates are presented in FIGS. 4A and 4B. TCRα and pTα proteins are readily visualized in cell lysates obtained from peripheral CD8⁺ T cells or TCRα-KO thymocytes, respectively. However, no evidence of either protein is present in FC lysates. In contrast, sequential TCRβ immunoprecipitation of the remaining FC lysate demonstrates the previously visualized 45 and 33 kD protein species of TCRβ and p33, thus assuring adequate sample quality and confirming the absence of TCRα and pTα chains in the CD3/TCRβ/p33 complex of this invention.

As demonstrated in Example Section 6, infra, the CD3/TCRβ/p33 complex of the invention correlates with the ability of FC to facilitate allogeneic stem cell engraftment. More specifically, when FC from mice deficient in TCRβ and therefore unable to express the p33 or CD3/TCRβ/p33 complex on their surface were utilized in bone marrow transplantation experiments with normal donor stem cells, stem cell engraftment failed. In addition, it was demonstrated that these deficient FC did not express the p33 protein on their surface. Thus, p33, TCRβ/p33 and/or the CD3/TCRβ/p33 complex of the invention play a central role in FC cell function including stem cell engraftment and the induction of donor-specific immunologic tolerance.

The p33 protein isolated and purified as described herein can be sequenced by standard protein sequencing techniques such as Edman degradation (see, e.g., Hewick et al., 1981, J. Biol. Chem. 256: 7990-7997) and its amino acid sequence determined. Using the amino acid sequence of the p33 protein, nucleic acid molecules encoding the protein can be obtained.

### 5.3. p33 Nucleic Acid Molecules of the Invention

The unique p33 protein sequence obtained as described above is used to deduce predicted gene sequences within the p33 gene, allowing the construction of synthetic oligonucleotide primers or probes having specificity for the p33 gene. These oligonucleotides are then used to screen gene libraries, e.g., cDNA or genomic libraries, from FC cells, which contain an array of DNA segments corresponding to FC genes. Those DNA sequences to which the oligonucleotide probes bind can then be sequenced, and using data from a variety of such p33 gene DNA sequences, the entire p33 gene sequence can be deduced. With the entire p33 gene sequence thus obtained, the p33 DNA sequences can be introduced into viral or phage vectors and transfected into desired host cells, e.g., cell lines, for a wide array of subsequent studies.

According to a preferred embodiment, the oligonucleotide probes derived from the p33 amino acid sequence are used to screen an expression DNA library constructed using subtraction cloning of T cell versus FC and thymocyte versus FC populations, in order to more selectively identify p33 gene candidates for subsequent screening. These expression DNA libraries are constructed using techniques well established in the art (see, e.g., Cho et al, 1998, Biochem. Biophys. Res. Comm. 242(1): 226-230 and Schraml et al., 1993, Trends in Genetics 9(3): 70-71). This approach allows analysis of only those FC proteins which are actively being produced (cDNA being transcribed) and are not present in T cells or thymocytes where p33 is not present.

Potential p33 DNA sequences identified in this way are then inserted into an expression vector, preferably a bacteriophage expression vector, containing a marker and transformed into a bacterial culture for subsequent colony screening. The colonies are screened using an anti-p33 antibody or by electrophoretic characteristics as defined for p33, e.g., kD, pI, etc., and/or optionally, using hybridization of secondary oligonucleotides that recognize other unique sites in the p33 cDNA. Following gene identification, those gene candidates which produce promising p33 protein products can be transfected into a murine T cell line lacking preTα and TCRα expression in order to study the expression and function of p33. A preferred T cell host is the TCRβ transgene of RAG-2 knockout mice as described by Shinkai et al., 1993, Science 259: 822, where the machinery for CD3/TCRβ expression is present when the appropriate associating protein, e.g., p33, TCRα or pTα, is expressed following introduction of the appropriate transgene.

According to another embodiment, where the p33 protein sequence is not utilized in the construction of oligonucleotides, p33 gene candidates can be selected using differential display comparing cDNA expression of FC with p33-negative cell populations such as T cells and thymocytes, e.g., by gel electrophoresis. This results in more initial sequences requiring insertion into the bacteriophage screening system but the subsequent screening by electrophoretic criteria and/or antibody would limit the number of possible candidates for subsequent murine expression and testing.

The p33 nucleic acid molecules obtained according to this invention include (a) any DNA sequence that encodes the amino acid sequence of the p33 protein isolated and purified as described supra; (b) any DNA sequence encoded by the cDNA or genomic clones obtained as described supra; and (c) any DNA sequence that hybridizes to the complement of DNA sequences (a) or (b) under highly stringent conditions, e.g., hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (see, e.g., Ausubel F. M. et al., eds., 1989, Current Protocols in Molecular Biology, Vol. I, Green Publishing Associates, Inc., and John Wiley & sons, Inc., New York, at p. 2.10.3) or under less stringent conditions, such as moderately stringent conditions, e.g., washing in 0.2×SSC/0.1% SDS at 42° C. (Ausubel et al., 1989, supra), and which encodes a gene product functionally equivalent to p33. “Functionally equivalent” as used herein refers to any protein capable of exhibiting a substantially similar in vivo or in vitro activity as p33, e.g., in enhancing stem cell engraftment.

As used herein, the term “p33 nucleic acid molecule” may also refer to fragments and/or degenerate variants of the above-identified DNA sequences, especially naturally occurring variants thereof. Such fragments include, for example, nucleotide sequences that encode portions of the p33 protein that correspond to functional domains of p33. Additionally, the p33 nucleic acid molecules of the invention include nucleic acid molecules, preferably DNA molecules, that hybridize under highly stringent or moderately stringent hybridization conditions to at least about 6, preferably at least about 12, and more preferably at least about 18, consecutive nucleotides of the DNA sequences of (a) or (b) identified supra. Also included within the p33 nucleic acid molecules of the invention are nucleic acid molecules, preferably DNA molecules, comprising a p33 nucleic acid, as described herein, operatively linked to a nucleotide sequence encoding a heterologous protein or peptide.

Moreover, due to the degeneracy of the genetic code, other DNA sequences which encode substantially the amino acid sequence of p33 may be used in the practice of the present invention for the cloning and expression of p33 polypeptides. Such DNA sequences include those which are capable of hybridizing to the p33 nucleic acids of this invention under stringent conditions, or which would be capable of hybridizing under stringent conditions but for the degeneracy of the genetic code.

Altered DNA sequences which may be used in accordance with the invention include deletions, additions or substitutions of different nucleotide residues resulting in a nucleic acid molecule that encodes the same or a functionally equivalent gene product. The gene product itself may contain deletions, additions or substitutions of amino acid residues within the p33 protein sequence, which result in a silent change, thus producing a functionally equivalent p33 polypeptide. Such amino acid substitutions may be made on the basis of similarity in polarity, charge, solubility, hydrophobicity, hydrophilicity, and/or the amphipatic nature of the residues involved. For example, negatively-charged amino acids include aspartic acid and glutamic acid; positively-charged amino acids include lysine and arginine; amino acids with uncharged polar head groups having similar hydrophilicity values include the following: leucine, isoleucine, valine; glycine, aniline; asparagine, glutamine; serine, threonine; phenylalanine, tyrosine. A functionally equivalent p33 polypeptide can include a polypeptide which enhances stem cell engraftment and/or induces donor-specific tolerance, but not necessarily to the same extent as its counterpart native p33.

The DNA nucleic acid molecules or sequences of the invention may be engineered in order to alter the p33 coding sequence for a variety of ends including but not limited to alterations which modify processing and expression of the gene product. For example, mutations may be introduced using techniques which are well known in the art, e.g., site-directed mutagenesis, to insert new restriction sites, to alter glycosylation patterns, phosphorylation, etc. For example, in certain expression systems such as yeast, host cells may over-glycosylate the gene product. When using such expression systems, it may be preferable to alter the p33 coding sequence to eliminate any N-linked glycosylation site. In another embodiment of the invention, the p33 nucleic acid or a modified p33 sequence may be ligated to a heterologous sequence to encode a fusion protein. The fusion protein may be engineered to contain a cleavage site located between the p33 sequence and the heterologous protein sequence, so that the p33 can be cleaved away from the heterologous moiety.

In an alternate embodiment of the invention, the coding sequence of p33 could be synthesized in whole or in part, using chemical methods well known in the art, based on the amino acid sequence of the p33 protein isolated as described herein. See, for example, Caruthers et al., 1980, Nuc. Acids Res. Symp. Ser. 7: 215-233; Crea and Horn, 1980, Nuc. Acids Res. 9(10): 2331; Matteucci and Caruthers, 1980, Tetrahedron Letters 21: 719; and Chow and Kempe, 1981, Nuc. Acids Res. 9(12): 2807-2817. Alternatively, the p33 protein itself could be produced using chemical methods to synthesize the p33 amino acid sequence in whole or in part. For example, peptides can be synthesized by solid phase techniques, cleaved from the resin, and purified by preparative high performance liquid chromatography (see, e.g., Creighton, 1983, Proteins Structures And Molecular Principles, W. H. Freeman and Co., N.Y., pp. 50-60). The composition of the synthetic peptides may be confirmed by amino acid analysis or sequencing (e.g., the Edman degradation procedure; see Creighton, 1983, Proteins, Structures and Molecular Principles, W. H. Freeman and Co., N.Y., pp. 34-49).

### 5.4. Expression of Recombinant p33 Polypeptides

The p33 nucleic acid molecules of the invention may be used to generate recombinant DNA molecules that direct the expression of p33 polypeptides, including the full-length p33 protein, functionally active or equivalent p33 peptides 4 thereof, or p33 fusion proteins in appropriate host cells.

In order to express a biologically active p33 polypeptide, a nucleic acid molecule coding for p33, or a functional equivalent thereof as described in Section 5.3, supra, is inserted into an appropriate expression vector, i.e., a vector which contains the necessary elements for the transcription and translation of the inserted coding sequence. The p33 gene products so produced, as well as host cells or cell lines transfected or transformed with recombinant p33 expression vectors, can be used for a variety of purposes. These include but are not limited to generating antibodies (i.e., monoclonal or polyclonal) that bind to the p33 protein, including those that competitively inhibit binding and “neutralize” p33 activity, and the screening and selection of p33 analogs.

Methods which are well known to those skilled in the art can be used to construct expression vectors containing the p33 coding sequences of the invention and appropriate transcriptional and translational control signals. These methods include in vitro recombinant DNA techniques, synthetic techniques and in vivo recombination/genetic recombination. See, for example, the techniques described in Maniatis et al., 1989, Molecular Cloning, A Laboratory Manual, Cold Spring Harbor Laboratory, N.Y. and Ausubel et al., 1989, Current Protocols in Molecular Biology, Greene Publishing Associates and Wiley Interscience, N.Y. See also Sambrook et al., 1989, Molecular Cloning, A Laboratory Manual, Cold Spring Harbor Press, N.Y.

A variety of host-expression vector systems may be utilized to express the p33 coding sequences of this invention. Such host-expression systems represent vehicles by which the coding sequences of interest may be produced and subsequently purified, but also represent cells which may, when transformed or transfected with the appropriate nucleotide coding sequences, exhibit the corresponding p33 gene products in situ and/or function in vivo. These include but are not limited to microorganisms such as bacteria (e.g., E. coli, B. subtilis) transformed with recombinant bacteriophage DNA, plasmid DNA or cosmid DNA expression vectors containing the p33 coding sequences; yeast (e.g., Saccharomyces, Pichia) transformed with recombinant yeast expression vectors containing the p33 coding sequences; insect cell systems infected with recombinant virus expression vectors (e.g., baculovirus) containing the p33 coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the p33 coding sequences; or mammalian cell systems (e.g., COS, CHO, BHK, 293, 3T3) harboring recombinant expression constructs containing promoters derived from the genome of mammalian cells (e.g., the metallothionein promoter) or from mammalian viruses (e.g., the adenovirus late promoter or vaccinia virus 7.5K promoter).

The expression elements of these systems vary in their strength and specificities. Depending on the host/vector system utilized, any of a number of suitable transcription and translation elements, including constitutive and inducible promoters, may be used in the expression vector. For example, when cloning in bacterial systems, inducible promoters such as pL of bacteriophage λ, plac, ptrp, ptac (ptrp-lac hybrid promoter) and the like may be used; when cloning in insect cell systems, promoters such as the baculovirus polyhedrin promoter may be used; when cloning in plant cell systems, promoters derived from the genome of plant cells (e.g., heat shock promoters; the promoter for the small subunit of RUBISCO; the promoter for the chlorophyll a/b binding protein) or from plant viruses (e.g., the 35S RNA promoter of CaMV; the coat protein promoter of TMV) may be used; when cloning in mammalian cell systems, promoters derived from the genome of mammalian cells (e.g., metallothionein promoter) or from mammalian viruses (e.g., the adenovirus late promoter; the vaccinia virus 7.5K promoter) may be used; when generating cell lines that contain multiple copies of the p33 DNA, SV40-, BPV- and EBV-based vectors may be used with an appropriate selectable marker.

In bacterial systems, a number of expression vectors may be advantageously selected depending upon the use intended for the p33 expressed. For example, when large quantities of p33 are to be produced for the generation of antibodies, vectors which direct the expression of high levels of fusion protein products that are readily purified may be desirable. Such vectors include but are not limited to the E. coli expression vector pUR278 (Ruther et al., 1983, EMBO J. 2: 1791), in which the p33 coding sequence may be ligated into the vector in frame with the lacZ coding region so that a hybrid p33/lacZ protein is produced; pIN vectors (Inouye & Inouye, 1985, Nucleic Acids Res. 13: 3101-3109; Van Heeke& Schuster, 1989, J. Biol. Chem. 264: 5503-5509); and the like. PGEX vectors may also be used to express foreign polypeptides as fusion proteins with glutathione S-transferase (GST). In general, such fusion proteins are soluble and can easily be purified from lysed cells by affinity chromatography, e.g., adsorption to glutathione-agarose beads followed by elution in the presence of free glutathione. The PGEX vectors are designed to include thrombin or factor Xa protease cleavage sites so that the cloned polypeptide of interest can be released from the GST moiety. See also Booth et al., 1988, Immunol. Lett. 19: 65-70; and Gardella et al., 1990, J. Biol. Chem. 265: 15854-15859; Pritchett et al., 1989, Biotechniques 7: 580.

In yeast, a number of vectors containing constitutive or inducible promoters may be used. For a review, see Current Protocols in Molecular Biology, Vol. 2, 1988, Ed. Ausubel et al., Greene Publish. Assoc. & Wiley Interscience, Ch. 13; Grant et al., 1987, Expression and Secretion Vectors for Yeast, in Methods in Enzymology, Eds. Wu & Grossman, 1987, Acad. Press, N.Y., Vol. 153, pp. 516-544; Glover, 1986, DNA Cloning, Vol. II, IRL Press, Wash., D.C., Ch. 3; and Bitter, 1987, Heterologous Gene Expression in Yeast, Methods in Enzymology, Eds. Berger & Kimmel, Acad. Press, N.Y., Vol. 152, pp. 673-684; and The Molecular Biology of the Yeast Saccharomyces, 1982, Eds. Strathern et al., Cold Spring Harbor Press, Vols. I and II.

In an insect system, Autographa californica nuclear polyhidrosis virus (AcNPV) can be used as a vector to express foreign genes. The virus grows in Spodoptera frugiperda cells. The p33 coding sequence may be cloned into non-essential regions (for example the polyhedrin gene) of the virus and placed under control of an AcNPV promoter (for example the polyhedrin promoter). Successful insertion of the p33 coding sequence will result in inactivation of the polyhedrin gene and production of non-occluded recombinant virus (i.e., virus lacking the proteinaceous coat coded for by the polyhedrin gene). These recombinant viruses can then be used to infect Spodoptera frugiperda cells in which the inserted gene is expressed (see e.g., Smith et al., 1983, J. Virol. 46: 584; Smith, U.S. Pat. No. 4,215,051).

In mammalian host cells, a number of viral-based expression systems may be utilized. In cases where an adenovirus is used as an expression vector, the p33 coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing p33 in infected hosts (see, e.g., Logan & Shenk, 1984, Proc. Natl. Acad. Sci. (USA) 81: 3655-3659). Alternatively, the vaccinia 7.5K promoter may be used (see, e.g., Mackett et al., 1982, Proc. Natl. Acad. Sci. (USA) 79: 7415-7419; Mackett et al., 1984, J. Virol. 49: 857-864; Panicali et al., 1982, Proc. Natl. Acad. Sci. 79: 4927-4931).

Specific initiation signals may also be required for efficient translation of inserted p33 coding sequences. These signals include the ATG initiation codon and adjacent sequences. In cases where the entire p33 gene, including its own initiation codon and adjacent sequences, is inserted into the appropriate expression vector, no additional translational control signals may be needed. However, in cases where only a portion of the p33 coding sequence is inserted, exogenous translational control signals, including the ATG initiation codon, must be provided. Furthermore, the initiation codon must be in phase with the reading frame of the p33 coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bitter et al., 1987, Methods in Enzymol. 153:516-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins. Appropriate cells lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERO, BHK, HeLa, COS, MDCK, 293, WI38, etc.

For long-term, high-yield production of recombinant proteins, stable expression is preferred. For example, cell lines which stably express the p33 polypeptides of this invention may be engineered. Rather than using expression vectors which contain viral origins of replication, host cells can be transformed with p33 nucleic acid molecules, e.g., DNA, controlled by appropriate expression control elements (e.g., promoter, enhancer, sequences, transcription terminators, polyadenylation sites, etc.), and a selectable marker. Following the introduction of foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express p33 on the cell surface. Such engineered cell lines are particularly useful in screening for p33 analogs or ligands.

In instances where the mammalian cell is a human cell, among the expression systems by which the p33 nucleic acid sequences of the invention can be expressed are human artificial chromosome (HAC) systems (see, e.g., Harrington et al., 1997, Nature Genetics 15: 345-355). p33 gene products can also be expressed in transgenic animals such as mice, rats, rabbits, guinea pigs, pigs, micro-pigs, sheep, goats, and non-human primates, e.g., baboons, monkeys, and chimpanzees. The term “transgenic” as used herein refers to animals expressing p33 nucleic acid sequences from a different species (e.g., mice expressing human p33 nucleic acid sequences), as well as animals that have been genetically engineered to overexpress endogenous (i.e., same species) p33 nucleic acid sequences or animals that have been genetically engineered to no longer express endogenous p33 nucleic acid sequences (i.e., “knock-out” animals), and their progeny.

Transgenic animals according to this invention may be produced using techniques well known in the art, including but not limited to pronuclear microinjection (Hoppe, P. C. and Wagner, T. E., 1989, U.S. Pat. No. 4,873,191); retrovirus mediated gene transfer into germ lines (Van der Putten et al., 1985, Proc. Natl. Acad. Sci., USA 82: 6148-6152); gene targeting in embryonic stem cells (Thompson et al., 1989, Cell 56: 313-321); electroporation of embryos (Lo, 1983, Mol Cell. Biol. 3: 1803-1814); and sperm-mediated gene transfer (Lavitrano et al., 1989, Cell 57: 717-723); etc. For a review of such techniques, see Gordon, 1989, Transgenic Animals, Intl. Rev. Cytol. 115: 171-229.

In addition, any technique known in the art may be used to produce transgenic animal clones containing a p33 transgene, for example, nuclear transfer into enucleated oocytes of nuclei from cultured embryonic, fetal or adult cells induced to quiescence (Campbell et al., 1996, Nature 380: 64-66; Wilmut et al., 1997, Nature 385: 810-813).

Host cells which contain the p33 coding sequence and which express a biologically active gene product may be identified by at least four general approaches; (a) DNA-DNA or DNA-RNA hybridization; (b) the presence or absence of “marker” gene functions; (c) assessing the level of transcription as measured by the expression of p33 mRNA transcripts in the host cell; and (d) detection of the gene product as measured by immunoassay or by its biological activity.

In the first approach, the presence of the p33 coding sequence inserted in the expression vector can be detected by DNA-DNA or DNA-RNA hybridization using probes comprising nucleotide sequences that are homologous to the p33 coding sequence, respectively, or portions or derivatives thereof.

In the second approach, the recombinant expression vector/host system can be identified and selected based upon the presence or absence of certain “marker” gene functions. For example, if the p33 coding sequence is inserted within a marker gene sequence of the vector, recombinants containing the p33 coding sequence can be identified by the absence of the marker gene function. Alternatively, a marker gene can be placed in tandem with the p33 sequence under the control of the same or different promoter used to control the expression of the p33 coding sequence. Expression of the marker in response to induction or selection indicates expression of the p33 coding sequence.

Selectable markers include resistance to antibiotics, resistance to methotrexate, transformation phenotype, and occlusion body formation in baculovirus. In addition, thymidine kinase activity (Wigler et al., 1977, Cell 11: 223) hypoxanthine-guanine phosphoribosyltransferase (Szybalska & Szybalski, 1962, Proc. Natl. Acad. Sci. USA 48: 2026), and adenine phosphoribosyltransferase (Lowy et al., 1980, Cell 22: 817) in tk⁻, hgprt⁻ or aprt⁻ cells, respectively. Also, antimetabolite resistance can be used as the basis of selection for dhfr, which confers resistance to methotrexate (Wigler et al., 1980, Proc. Natl. Acad. Sci. USA 77: 3567; O'Hare et al., 1981, Proc. Natl. Acad. Sci. USA 78: 1527); gpt, which confers resistance to mycophenolic acid (Mulligan & Berg, 1981, Proc. Natl. Acad. Sci. USA 78: 2072); neo, which confers resistance to the aminoglycoside G-418 (Colberre-Garapin, et al., 1981, J. Mol. Biol. 150: 1); and hygro, which confers resistance to hygromycin (Santerre et al., 1984, Gene 30: 147). Additional selectable genes have been described, namely trpB, which allows cells to utilize indole in place of tryptophan; hisD, which allows cells to utilize histinol in place of histidine (Hartman & Mulligan, 1988, Proc. Natl. Acad. Sci. USA 85: 8047); and ODC (ornithine decarboxylase) which confers resistance to the ornithine decarboxylase inhibitor, 2-(difluoromethyl)-DL-ornithine, DFMO (McConlogue, 1987, in Current Communications in Molecular Biology, Cold Spring Harbor Laboratory ed.).

In the third approach, transcriptional activity for the p33 coding region can be assessed by hybridization assays. For example, RNA can be isolated and analyzed by Northern blot using a probe homologous to the p33 coding sequence or particular portions thereof. Alternatively, total nucleic acids of the host cell may be extracted and assayed for hybridization to such probes.

In the fourth approach, the expression of the p33 protein product can be assessed immunologically, for example by Western blots, immunoassays such as radioimmuno-precipitation, enzyme-linked immunoassays and the like. The ultimate test of the success of the expression system, however, involves the detection of biologically active p33 gene product. A number of assays can be used to detect p33 activity including but not limited to TCRβ and/or CD3∈ binding assays and biological assays for stem cell engraftment.

Once a clone that produces high levels of a biologically active p33 polypeptide is identified, the clone may be expanded and used to produce large amounts of the polypeptide which may be purified using techniques well known in the art, including but not limited to, immunoaffinity purification using antibodies, immunoprecipitation or chromatographic methods including high performance liquid chromatography (HPLC).

Where the p33 coding sequence is engineered to encode a cleavable fusion protein, purification may be readily accomplished using affinity purification techniques. For example, a collagenase cleavage recognition consensus sequence may be engineered between the carboxy terminus of p33 and protein A. The resulting fusion protein may be readily purified using an IgG column that binds the protein A moiety. Unfused p33 may be readily released from the column by treatment with collagenase. Another example would be the use of PGEX vectors that express foreign polypeptides as fusion proteins with glutathionine S-transferase (GST). The fusion protein may be engineered with either thrombin or factor Xa cleavage sites between the cloned gene and the GST moiety. The fusion protein may be easily purified from cell extracts by adsorption to glutathione agarose beads followed by elution in the presence of glutathione. In fact, any cleavage site or enzyme cleavage substrate may be engineered between the p33 gene product sequence and a second peptide or protein that has a binding partner which could be used for purification, e.g., any antigen for which an immunoaffinity column can be prepared.

In addition, p33 fusion proteins may be readily purified by utilizing an antibody specific for the fusion protein being expressed. For example, a system described by Janknecht et al. allows for the ready purification of non-denatured fusion proteins expressed in human cell lines (Janknecht, et al., 1991, Proc. Natl. Acad. Sci. USA 88: 8972-8976). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺.nitriloacetic acid-agarose columns and histidine-tagged proteins are selectively eluted with imidazole-containing buffers.

### 5.5. ANTIBODIES TO p33 POLYPEPTIDES

The present invention also provides for methods for the production of antibodies directed to the p33 polypeptides of this invention, including antibodies that specifically recognize one or more p33 epitopes or epitopes of conserved variants or peptide fragments of p33.

Such antibodies may include, but are not limited to, polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above. Such antibodies may be used, for example, in the detection of a p33 protein or polypeptide in an biological sample and may, therefore, be utilized as part of a diagnostic or prognostic technique whereby patients may be tested for abnormal levels of p33, and/or for the presence of abnormal forms of the protein. Such antibodies may also be utilized in conjunction with, for example, compound screening protocols for the evaluation of the effect of test compounds on p33 levels and/or activity. Additionally, such antibodies can be used in conjunction with the gene therapy techniques described in Section 5.6, infra, to, for example, evaluate the normal and/or genetically-engineered p33-expressing cells prior to their introduction into the patient.

For the production of antibodies against p33, various host animals may be immunized by injection with the protein or a portion thereof. Such host animals include rabbits, mice, rats, hamsters and baboons. Various adjuvants may be used to increase the immunological response, depending on the host species, including but not limited to, TiterMax Gold adjuvant (CytRx Corp., Norcross Ga.), Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as p33, or an antigenic functional derivative thereof. For the production of polyclonal antibodies, host animals such as those described above, may be immunized by injection with p33 supplemented with adjuvants as also described above.

Monoclonal antibodies, which are homogeneous populations of antibodies to a particular antigen, may be obtained by any technique which provides for the production of antibody molecules by continuous cell lines in culture. These include, but are not limited to, the hybridoma technique of Kohler and Milstein (1975, Nature 256: 495-497; and U.S. Pat. No. 4,376,110), the human B-cell hybridoma technique (Kosbor et al., 1983, Immunology Today 4: 72; Cole et al., 1983, Proc. Natl. Acad. Sci. USA 80: 2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridomas producing the monoclonal antibodies of this invention may be cultivated in vitro or in vivo.

In addition, techniques developed for the production of “chimeric antibodies” (Morrison et al., 1984, Proc. Natl. Acad. Sci., 81: 6851-6855; Neuberger et al., 1984, Nature 312: 604-608; Takeda et al., 1985, Nature 314: 452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region (see, e.g., Cabilly et al., U.S. Pat. No. 4,816,567; and Boss et al., U.S. Pat. No. 4,816,397.)

In addition, techniques have been developed for the production of humanized antibodies (see, e.g., Queen, U.S. Pat. No. 5,585,089). Humanized antibodies are antibody molecules from non-human species having one or more CDRs from the non-human species and a framework region from a human immunoglobulin molecule.

Alternatively, techniques described for the production of single chain antibodies (U.S. Pat. No. 4,946,778; Bird, 1988, Science 242: 423-426; Huston et al., 1988, Proc. Natl. Acad. Sci. USA 85: 5879-5883; and Ward et al., 1989, Nature 334: 544-546) can be used in the production of single chain antibodies against p33. Single chain antibodies are formed by linking the heavy and light chain fragments of the Fv region via an amino acid bridge, resulting in a single chain polypeptide.

Furthermore, antibody fragments which recognize specific epitopes of p33 may be produced by techniques well known in the art. For example, such fragments include but are not limited to, F(ab′)₂ fragments which can be produced by pepsin digestion of the antibody molecule and Fab fragments which can be generated by reducing the disulfide bridges of the F(ab′)₂ fragments. Alternatively, Fab expression libraries may be constructed (Huse et al., 1989, Science 246: 1275-1281) to allow rapid and easy identification of monoclonal Fab fragments with the desired specificity.

### 5.6. Uses of the p33 Nucleic Acid Molecules, Gene Products, and Antibodies

The p33 polypeptides of this invention are useful for promoting hematopoeitic stem cell engraftment and donor-specific tolerance for the enhancement of transplantation success or outcomes. The promotion of stem cell engraftment and tolerance is important not only in organ or tissue transplantation, i.e., to promote acceptance of the organ or tissue by the transplant recipient, but in the treatment of leukemias and other hematological diseases which require bone marrow transplantation and in which the transplanted bone marrow must be accepted by the recipient patient.

Thus, the present invention includes methods of promoting stem cell engraftment and donor-specific tolerance for the enhancement of organ or tissue transplantation success as well as methods of promoting stem cell engraftment and/or donor-specific tolerance in bone marrow transplantation in the treatment of leukemia and hematological disease.

The p33 polypeptides of the invention may be useful in the form of the isolated protein or polypeptide, or as part of a TCRβ/p33 or CD3/TCRβ/p33 complex, preferably expressed on the surface of a cell. For example, the p33 polypeptides or complexes of the invention can be introduced into donor organs or tissues by gene therapy or transgenic procedures, thus enabling the donor organs or tissues to express the p33 polypeptides or complexes on their cell surfaces, and thus promoting engraftment and immunologic tolerance in the transplant patient. When desiring expression of the TCRβ/p33 or CD3/TCRβ/p33 complex on the donor cells, the DNA sequences for CD3 and/or TCRβ must be introduced into the cells in addition to the p33 DNA sequences. DNA sequences for CD3 and TCRβ and their successful transfection into host cells are known in the art (see, e.g., Kishi et al., 1991, EMBO J. 10(1): 93-100). According to another embodiment, a cell population may be turned into an “FC” surrogate by genetically engineering p33, TCRβ/p33 or CD3/TCRβ/p33 complex expression on a “non-FC” cell.

Alternatively, hematopoeitic stem cells from the transplant donor can be genetically engineered as described supra to express p33 or a TCRβ/p33 or CD3/TCRβ/p33 complex of the invention, thus promoting stem cell engraftment and donor-specific tolerance in connection with transplantation without the need for the concurrent administration of FC cells.

According to another embodiment, it may be possible to activate or otherwise modify donor stem cells in vitro by incubation with p33 polypeptides prior to transplantation.

Alternatively, the p33 polypeptides of the invention may be administered in a pharmaceutical composition, alone or in complex with TCRβ and/or CD3. In addition, pharmaceutical compositions of the invention can include cellular compositions comprising genetically-engineered cell populations having p33 or TCRβ/p33 or CD3/TCRβ/p33 on their surface. The pharmaceutical compositions of the invention can be administered to a patient at therapeutically effective doses to enhance donor cell/tissue/organ engraftment and/or to induce donor-specific immunologic tolerance in vivo. A therapeutically effective dose refers to that amount of the compound or cell population sufficient to produce the desired engraftment or tolerance. Moreover, toxicity and therapeutic efficacy can be determined by standard pharmaceutical procedures, e.g., in cell culture or experimental animals. For example, LD₅₀, the dose lethal to 50% of the population, or ED₅₀ the dose therapeutically effective in 50% of the population, can be determined by standard methods known in the art.

Thus, the data obtained from cell culture assays or experimental animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized.

The pharmaceutical compositions of the invention can be formulated in conventional manner using one or more physiologically acceptable carriers or excipients. Thus, the compounds and their physiologically acceptable salts and solvents can be formulated for administration by inhalation or insufflation (either through the mouth or the nose) or oral, buccal, parenteral or rectal administration.

For oral administration, the pharmaceutical compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound. For buccal administration the compositions can take the form of tablets or lozenges formulated in conventional manner.

The compounds can be formulated for parenteral administration (i.e., intravenous or intramuscular) by injection, via, for example, bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

In addition to the formulations described previously, the compounds can also be formulated as a depot preparation. Such long acting formulations can be administered by implantation (for example, subcutaneously or intramuscularly) or by intramuscular injection. Thus, for example, the compounds can be formulated with suitable polymeric or hydrophobic materials (for example, as an emulsion in an acceptable oil) or ion exchange resins, or as sparingly soluble derivatives, for example, as a sparingly soluble salt.

The p33 nucleic acids of the invention are useful for the efficient production and purification of p33 polypeptides and for use in methods for introducing p33 gene products and hence expression into desired cells or tissues, e.g., for transplantation in vivo.

The p33 antibodies of this invention are useful for methods for detecting, isolating or purifying the p33 polypeptides of the invention. For example, the p33 antibodies are useful for the efficient isolation and purification of p33 polypeptides for any of the uses immediately above. These antibodies may also be used as diagnostic tools, e.g., in in vitro assays to determine the level of p33 expression in cells that have been genetically engineered to produce and/or express p33 or its complex. The antibodies of this invention may also be used to quantitatively or qualitatively detect the present of p33 gene products in a sample or on a cell surface, including their use histologically, e.g., in immunofluorescence or immunoelectron microscopy for in situ detection of p33 polypeptides. In addition, the p33 antibodies of the invention may be useful for therapeutic applications.

### 6. Example: Isolation and Characterization of the p33 Protein of the Invention

This section describes the isolation, identification, purification and characterization of the p33 protein of the invention.

### 6.1. Isolation of Facilitating Cells

FC were isolated from normal bone marrow of C57BL/6J (B6) mice via live, sterile, rare-event multiparameter cell sorting (FACStar Plus, Becton Dickenson Immunocytometry Systems, San Jose Calif.). The bone marrow (BM) was isolated and resuspended at a concentration of 70-150×10⁶ cells/ml in sterile cell sort media (CSM), which consisted of sterile Hanks' Balanced Salt Solution without phenol (Gibco, Grand Island N.Y.), 2% heat-inactivated fetal calf serum (FCS; Summit Biotech, Fort Collins Colo.), 2 μl/ml HEPES buffer (Gibco), and 150 mg/ml Gentamicin (Gibco). Directly-labeled monoclonal antibodies, αβTCR-FITC [H57-597], γδTCR-FITC [GL3] and CD8-PE [53-6.7] (Pharmingen, San Diego Calif.), were added at saturating concentrations, and the cells were incubated for 45 minutes. The BM cells were washed twice with CSM, aliquotted into 12×75 mm tubes (Falcon) and subjected to cell sorting. FC were isolated from the lymphoid gate as CD8-PE⁺ and αβ and γδTCR-FITC^{dim/-} cells, and were collected into 1 ml of CSM for subsequent analysis. The T cells used in these experiments were isolated from the spleens of B6 mice and sorted as described above and isolated from the lymphoid gate as CD8-PE⁺ and αβ and γδTCR-FITC^{bright} cells and collected for subsequent analysis. The thymocytes used in these experiments were derived from the thymus of TCRα KO mice. Cell populations that were less than 90% pure on post-sort analysis were not used in experiments.

### 6.2. Surface-Biotinylation of Fc Cells and Immunoprecipitation of p33 Complex

Prior to biotinylation, the FC were washed twice in serum-free phosphate-buffered saline (PBS) to remove soluble contaminating proteins. Surface protein biotinylation was then performed on the FC and T cell populations utilizing Sulfo-N-Hydroxy succinimidester-LC-Biotin (Pierce, Rockford, Ill.) according to the methods described by Altin et al., 1995, Anal. Biochem. 224: 382-389 and Meier et al., 1992, Anal. Biochem. 204: 220-226. The biotinylated cells were then lysed at 1×10⁶ cells/ml in lysis buffer containing 1% Digitonin (Boehringer Mannheim, Indianapolis Ind.), 150 mmol NaCl, 20 mmol Tris-HCl (pH=7.6) and protease inhibitors (PMSF, aprotinin and leupeptin).

Immunoprecipitation was then carried out by adding 2-5 mg/ml of monoclonal antibody directed against CD3∈, TCRA, TCRβ (all from Pharmingen) or pTα (kindly provided by Wiest et al., Fox Chase Cancer Center, Philadelphia, Pa.) to the cell lysates for one hour. Immune complexes were precipitated by adding 20 μl Protein G sepharose (Amersham Pharmacia Biotech, Piscataway N.J.) to each sample with incubation for three hours on a roller at 4° C. For the pTα studies, 20 μl of Protein A sepharose was used for immunoprecipitation. Control immunoprecitations were routinely carried out using an irrelevant isotype control monoclonal antibody. For serial immunoprecipitation studies, the lysates remaining after the initial immunoprecipitation were treated with the desired second antibody and complexes were precipitated as described above.

### 6.3. Isolation of the p33 Protein and/or CD3/TCRβ/p33 Complex

Next, the immunoprecipitated proteins were separated by one-dimensional SDS-PAGE as follows: 20 μl of reducing or non-reducing SDS Sample Buffer (4% SDS, 20% Glycerol, 0.125 M Tris-HCl (pH 6.8), 0.05% Bromophenol Blue ±10% 2-mercaptoethanol) was added to the protein-sepharose pellets derived from the prior immunoprecipitation, and the samples were boiled for 5 minutes. After a brief spin in the microcentrifuge, the supernatant/eluate was loaded onto 10% (for non-reducing studies) and 12.5% (for reducing studies) 0.75 mm polyacrylamide gels. Gels were run at a constant amperage (20 mA/gel; 100-400V) until the dye front reached the bottom of the gel.

Western blots were then performed in Carbonate Transfer Buffer (10 mM NaHCO₃, 3 mM Na₂CO₃ and 15% v/v methanol, (pH 9.9)). For two hours at 200 mA; 22-27V and the membranes (PVDF membranes, Micron Separations, Inc., Westborough Mass.) were blocked for one hour in Membrane Blocking Buffer (PBS, 0.1% Tween 20, 5% nonfat dry milk), and then washed three times for 5 minutes in PBS/T wash buffer (PBS, 0.1% Tween 20). The blots were then incubated in 50-100 ml of a 1:5,000-1: 20,000 dilution of streptavidin-horseradish peroxidase conjugate (Pierce). The membranes were then washed five times for five minutes in PBS/T, incubated in the ECL (Enhanced Chemiluninescence) detection reagent, SuperSignal® (Pierce) for 5 minutes and then exposed to film for 1-45 minutes.

The SDS-PAGE and Western blotting of the immunoprecipitates under reducing conditions allowed the detection and isolation of the desired p33 protein as well as any associated proteins on the FC cell surface.

### 6.4. Results

As demonstrated in FIG. 2A, immunoprecipitation using a monoclonal antibody directed to CD3∈ and Western blotting under nonreducing conditions, demonstrated the presence of the 25 kD CD3 protein in both the FC and T cell lanes, indicating that CD3 is present on both FC and T cell populations. An 85 kD protein band was also noted in the T cell lane, which is consistent with the TCRα/TCRβ disulfide-linked heterodimer known to be present on T cells. Furthermore, subsequent analysis of this 85 kD protein, i.e., under reducing conditions, indicated that it is composed of a 40 kD and a 45 kD protein corresponding to the TCRα and TCRβ proteins, respectively.

In contrast, CD3 expression in the FC population was associated with a 75 kD band not observed in the T cell lane. In subsequent analysis of the FC lysates, i.e., under reducing conditions, the 75 kD complex was separated into two distinct protein bands of 45 and 33 kD (see FIG. 2B). The protein band at 33 kD represents the isolated p33 protein of this invention. Furthermore, these results suggested that an alternative heterodimer is associated with CD3 on FC cells.

In addition, immunoprecipitation and Western blotting utilizing a monoclonal antibody specific for TCRβ (H57-597) and under reducing conditions performed on biotinylated FC and T cell lysates yielded the identical 45 and 33 kD protein bands detected in the CD3 immunoprecipitation studies described supra, confirming that the identity of the CD3-associated 45 kD protein on the FC surface is, in fact, TCRβ (see FIG. 2C). In contrast, serial immunoprecipitation experiments with TCRα or pTα antibodies followed by TCRβ immunoprecipitation on FC, T cell or thymocyte cell lysates demonstrated that TCRα or pTα, while expressed on T cells and thymocytes, are not expressed on the FC cell surface and do not form part of the CD3/TCR/p33 complex on FC (see FIGS. 4A and 4B). Furthermore, immunoprecipitations performed with anti-γδTCR antibody, GL3, did not yield 45 kD or 33 kD proteins from FC lysates, suggesting that the 45 kD and 33 kD proteins on FC do not represent. γδ heterodimers (data not shown). Finally, the coprecipitation of the FC-associated 33 kD protein with both CD3 and TCRβ strongly suggests that these three molecules are associated as a single complex on the FC cell surface.

### 6.5. Characterization of p33

Next, nonreducing and reducing two-dimensional electrophoresis was performed after immunoprecipitation of surface-biotinylated proteins from FC and T cell lysates with CD3∈ monoclonal antibody. Samples were run initially under non-reducing discontinuous Laemmli SDS-PAGE conditions. Lanes were excised and equilibrated in 10 ml of reduced SDS sample buffer under mild agitation at room temperature for 30 minutes. The gel strip was then transferred onto a second 12.5% acrylamide gel. The strip was fixed with heated SDS sample buffer mixed with 1% agarose. Electrophoresis and Western blotting were carried out as described supra.

As depicted in FIG. 3, FC possess a CD3-associated ˜78 kD dimer in the non-reduced dimension that departs from the diagonal after reduction and separates into 45 kD (TCRβ) and 33 kD (p33) proteins. The expected 45 kD (TCRβ) and 40 kD (TCRα) kD proteins of the TCR heterodimer present in the T cell lysates are also shown for comparison. These experiments suggest that TCRβ and p33 exist as a disulfide-linked heterodimer, which is noncovalently associated with CD3 on the surface of the FC.

The p33 protein of the invention was further characterized by isoelectric focusing (see, e.g., O'Farrell et al., 1977, Cell 12: 1133-1142) and glycosidase digestion studies as follows:

Isoelectric focusing gels were created by mixing a solution composed of 2.19 g urea, 0.42 ml of acrylamide stock solution (30% acrylamide, 5.7% methylene-bisacrylamide), 0.82 ml of 10% NP-40, 0.89 ml of dH₂O and 0.2 ml of ampholytes (pH 3-10) at room temperature until the urea dissolves. Ammonium Persulfate (25 μl of 10% stock) and 2.5 ml of TEMED were added and the gel solution was poured into the minigel casting chamber (Hoefer Scientific Instruments, San Francisco Calif.). Polymerization was complete after 45 minutes. Immunoprecipitates were eluted utilizing a reducing Elution Buffer (9.5M urea, 2% Nonidet P-40, 2% ampholytes, 10% 2-mercaptoetanol) via thorough vortexing at room temperature for 15 minutes. The beads were spun down, and the supernatant/eluate from each sample was added to the top of each isoelectric focusing tube. The tubes were then loaded into the-IEF apparatus (Hoefer). Catholyte Solution (20 mM arginine, 20 mM lysine in water; pH˜10.2) was added to the upper chamber and the system was inspected for leaks. Anolyte Solution (8 mM phosphoric acid in water) was then added to the lower chamber. Sample overlay solution (5 μl) was added to the top of each tube and pre-run for 30 minutes at 200 V. After the samples were added, the separation run was performed for 3 hours at 400 V. End-of-run focusing was performed for 30 minutes at 500 V. At the conclusion of isoelectric focusing, the tube gels were extruded from the glass tubes under gentle syringe pressure. The gels underwent equilibration for 30 minutes at room temperature in Equilibration Buffer (2.3% SDS, 10% Glycerol, 62.5 mM Tris-HCl (pH 6.8), 0.05% Bromophenol Blue ±10% 2-mercaptoethanol). The tube gels were then loaded onto the second dimension 12.5% SDS-PAGE 1.5 mm acrylamide gel and run at 30 mA, 150-350 V until the dye front reached the bottom of the gel.

Peptidyl-N-Glycosidase F (PNGase F) digestion was performed as follows: Immunoprecipitated protein-sepharose pellets were taken up in 20 μl PNGase F buffer (250 mM Na₃PO₄, 50 mM EDTA, 0.5% SDS, 1% 2-mercaptoethanol (pH 8.0), vortexed and boiled for 5 minutes. A 1.7% NP-40 solution (30 μl) was added and vortexed. The sample was divided into two. To one portion of the sample, 0.5 μl of a 1000 U/ml stock of PNGase F solution (Sigma, St. Louis Mo.) was added and mixed. The other portion of the sample received no PNGase F. Both portions were incubated at 37° C. for 10 hours. The samples were then boiled in 15 μl 4× SDS-PAGE sample buffer and loaded onto 12.5% SDS-polyacrylamide gels.

The isoelectric focusing studies readily identified the p33 protein within the FC sample at a rMW of 33 kD and an apparent isoelectric point of 4.5 (see FIG. 5A). In addition, p33 undergoes N-linked glycosylation, as demonstrated by the reduction in molecular mass in the presence of the PNGase F enzyme (see FIG. 5B).

The results of the above-described experiments demonstrate that the p33 protein of this invention represents a biochemically distinct CD3/TCRβ-associated glycoprotein (see Table I, supra).

### 6.6. The Presence of the CD3/TCRβ/p33 Complex Correlates With the Ability to Facilitate Allogeneic Stem Cell Engraftment

To determine whether the novel CD3/TCRβ/p33 complex of the present invention plays an important role in the promotion of allogeneic stem cell engraftment, putative FC populations (CD8⁺/TCR^{dim/-}) were isolated from the bone marrow of TCRβ and RAG-1 knockout mice (Jackson Laboratory, Bar Harbor, Me.). These mice are unable to produce TCRβ, and are therefore unable to express a critical component of the CD3/TCRβ/p33 complex. If this complex does indeed play an important role in FC function, deficiency of a critical component should prevent cells with the CD8⁺/TCR^{dim/-} phenotype from facilitating allogeneic stem cell engraftment.

Therefore, lethally-irradiated B10. BR recipients were reconstituted with 10,000 stem cells derived from normal B6 donors together with 30,000 CD8⁺/TCR^{dim/-} FC sorted from the BM of normal B6 (TCRβ^{+/+}), TCRβ-knockout (TCR^{-/-}) and RAG-knockout donors. More specifically, purified stem cells were isolated from four to six week old male B6 mice via sterile, rare-event, multiparameter cell sorting as Stem Cell Antigen⁺/c-kit⁺/Lineage⁻ (Lineage: CD8, αβTCR, GR-1, MAC-1, B220). FC populations were sorted as CD8⁺/αβ and γδTCR^{dim/-} from B6 mice as controls, or from B6 strains deficient in TCRβ expression due to inactivation of TCRβ (C57BL/6J-Tcrb^{tmlMom}; TCRβ^{-/-}) and Recombination Activating Gene-1 (C57BL/6JRag1^{tmlMom}; RAG1-KO or RAG1^{-/-}). Animals were housed in a specific pathogen-free facility at the Pittsburgh Cancer Institute (University of Pittsburgh Medical Center, Pittsburgh, Pa.).

10,000 purified stem cells were administered alone or in combination with 30,00-50,000 purified FC after suspension in 1 ml of Medium 199 (Gibco). Intravenous injections were performed via the lateral tail vein in lethally-irradiated (950 rads) B10.BR (B10.BRSgSnJ purchased from Jackson Laboratory) recipient mice. Animals were monitored daily and peripheral blood lymphocyte typing was performed routinely at 1, 3, and 6 months to evaluate the extent of donor cell engraftment. Stem cell integrity was documented in each experiment by long-term survival (3 months) following syngeneic transplantation of as few as 1,000 stem cells. Recipients were monitored for engraftment and survival for a period of 6 months.

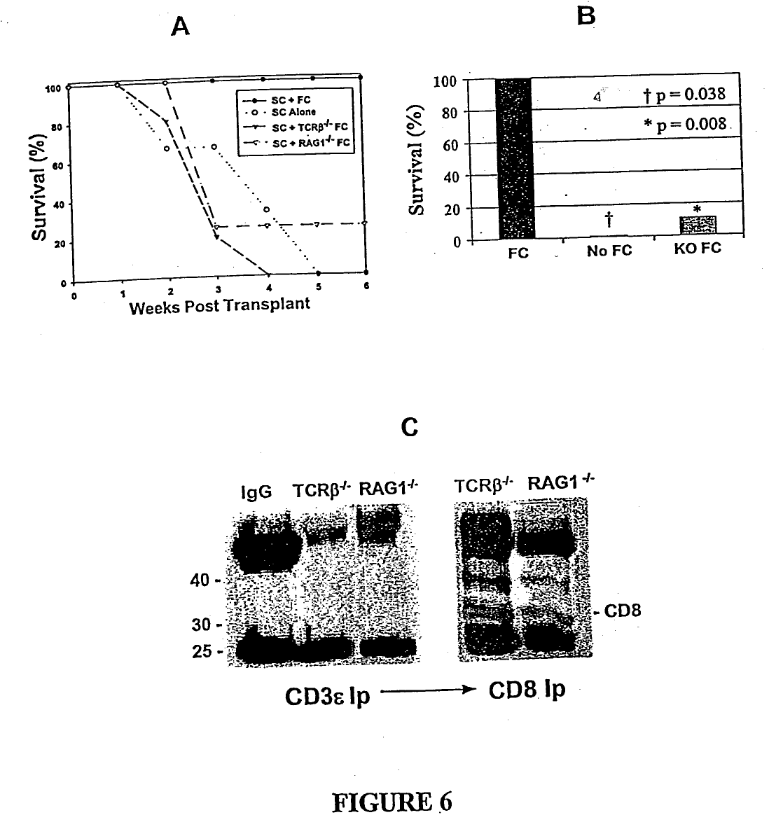

As demonstrated in FIG. 6A, transplantation of 10,000 normal B6 stem cells alone fail to reconstitute recipients across complete MHC Class I and Class II barriers (SC_{B6}→B10.BR). Engraftment failure did not occur with the addition of 30,000 FC from normal B6 donors, resulting in long-term survival and donor chimerism in all fully allogeneic recipients (SC_{B6}+FC_{B6}→BR; n=5). In contradistinction, administration of 30,000 CD8⁺/TCR^{dim/-} FC from RAG^{-/-} or TCRβ^{-/- donors failed to promote stem cell engraftment, and all recipients of SC}_{B6} and FC_{knockout} transplants succumbed to radiation-induced aplasia (n=5 per group) (see FIG. 6B). The single FC_{knockout} recipient that survived greater than one month following transplantation exhibited a significant degree of syngeneic reconstitution, which eventually failed.

In contrast, all B6 stem cell+FC B6 recipients exhibited evidence of donor engraftment as assessed by peripheral blood typing between four and six weeks following reconstitution. Despite an identical flow cytometric phenotype of CD8+/TCRαβ−/TCRγδ− for FC isolated from control B6 and RAG^{-/- or TCRβ}^{-/-} donors, recipient survival in the FC_{knockout} groups was no different than administration of stem cells alone (see FIG. 6B). Furthermore, CD3∈ immunoprecipitation carried out on these FC knockout lysates confirmed that p33 is not expressed on the surface of these putative FC (see FIG. 6C), despite the presence of CD8+ cells in the FC samples. The ability of the isolated FC populations to facilitate allogeneic stem cell engraftment thus directly correlated with the expression of the CD3/TCRβ/p33 complex on the FC surface. Without this complex, the FC effect is completely lost. These results suggest that the CD3/TCRβ/p33 complex expressed on the FC population plays a central role in FC function.

### 7. Discussion

The T cell receptor (TCR) is composed of six distinct, type I transmembrane polypeptides (Weissman, 1994, Chem. Immunol. 59: 1-18). On mature T cells, these subunits consist of clonotypic TCRα/β (or TCRγ/δ) heterodimers (Dembic et al., 1986, Nature 320: 232; Saito et al., 1987, Nature 325: 125) noncovalently associated with invariant CD3δ∈ and γ∈ heterodimers (Marrack et al., 1987, Science 238: 1073) and CD3ζ-ζ (or ζ−n) dimers (Blumberg et al., 1991, Eur. J. Immunol. 21: 473-481). While the clonotypic TCR heterodimers provide antigen/MHC specificity, the CD3 subunits mediate intracellular signal transduction (Clevers et al., 1988, Ann. Rev. Immunol. 6: 629-662). Only completely-assembled, functional complexes gain access to the cell surface. Reports describing T cell variants that are unable to produce CD3γ (Geisler, 1992, J. Immunol. 148: 2437), CD3δ (Buferne et al., 1992, J. Immunol. 148: 657), CD3δ (Wang et al., 1999, J. Immunol. 162: 88-94), CD3ζ (Sussman et al., 1988, Cell 52: 85-95) or TCRβ (Chen et al., 1988, J. Cell. Biol. 107: 2149-2161) convincingly demonstrate that simultaneous production of each of these components is critical for normal assembly and surface expression of such TCR complexes. Partial receptors and unassembled subunits are retained in the ER, or are targeted for lysosomal degradation (Lippincott-Schwartz et al., 1988, Cell 54: 209-220; Klausner et al., 1990, Ann. Rev. Cell. Biol. 6: 403-431).

In contrast, the expression of CD3 without conventional TCR heterodimers has been well-documented, most notably in immature thymocytes (Ley et al., 1989, supra; Groettrup et al., 1993a, 1993b, supra; Wiest et al., 1994, 1995, supra). In all four reported cases, CD3 joins with TCRβ and/or an additional stabilizing or chaperone protein to promote receptor stability and confer functional specificity (Wiest et al., 1994, 1995, supra). First, CD3 associates with a TCRβ dimer in a small subset of fetal thymocytes (Groettrup et al., 1993a, supra), as well as on thymocytes of TCRβ-deficient mice and immature T cell lines transfected with a productively rearranged TCRβ gene (Kishi et al., 1991, supra; von Boehmer et al., 1998, supra). Second, CD3 is expressed on immature thymocytes as part of a clonotypic-independent complex with the 90 kD molecular chaperone calnexin (Wiest et al., 1995, supra). Third, CD3/TCRβ complexes associate with a TCRδ chain in T leukemia cell lines and in developing thymocytes unable to rearrange the TCRα locus (Hochstenbach et al., 1989, Nature 340 (6234): 562-565). Lastly, CD3 expression in the absence of conventional TCR heterodimers is seen in the Pre-T cell receptor, which combines CD3 with TCRβ and a 33 kD glycoprotein, pTα (Groettrup et al., 1993b, supra). All of these CD3-associated complexes are capable of supporting CD3 signal transduction, cellular activation and developmental progression from the CD4⁻CD8⁻ (DN) to the CD4⁺CD8⁺ (DP) phase of thymocyte development. In addition, the relative expression of CD3 (and TCRβ) in all of these receptor complexes is found to be somewhat lower than that seen on mature T cells by flow cytometry (Jacobs et al., 1994, Eur. J. Immunol. 24: 934-939.; Wiest et al., 1994, supra). This lower level of CD3 expression is also characteristic of the CD8⁺/CD3⁺ FC population. Thus, the CD3/TCRβ/p33 complex described herein might represent a member of an emerging CD3/TCRβ “family” of receptors, characterized by dim expression of CD3 and TCRβ in association with various TCRα surrogate proteins, all of which have unique and potent biological activities that differ from those of the bright CD3⁺ mature T cells.

Moreover, p33 may represent one of a family of proteins that enhance stem cell engraftment, i.e., SEEP proteins, which proteins are present on a subset of CD8⁺ cells lacking conventional TCRαβ heterodimer expression. For example, the pTα is an approximately 33 kD protein that is expressed on the surface of developing T cells in the thymus, but not on peripheral T cells and it associates with CD3 and TCRβ (similar to p33 on FC cells). pTα has also been described as laying an important role in the selection of developing T cells for the αβTCR lineage, promoting TCRβ allelic exclusion and expansion of CD4+CD8+ cells. It has also been shown to play a role in positive selection of developing thymocytes. Since the thymus and thymocytes themselves have been implicated in tolerance induction to solid organ and cellular grafts, it is possible that pTα may play a critical role in the molecular events associated with immunologic tolerance and thus may represent another SEEP protein as defined herein. Thus, p33 may represent one of a number of stem cell engraftment-enhancing proteins that promote the engraftment of allogeneic donor cells in transplant recipients and induce immunologic tolerance to donor cells and tissues in those recipients.

TCR chains associate with Type I transmembrane molecules via a disulfide-linkage. Since all CD3/TCRβ-associated proteins identified to date form this type of disulfide linkage and without these linkages, little or no functional TCR-CD3 complexes can be expressed (Sancho et al., 1989, J. Biol. Chem. 264: 20760; Minami et al., 1987, Proc. Natl. Acad. Sci. USA 84: 2688-2692, 1987), it is suggested that the structural organization of the CD3/TCRβ/p33 complex is similar in configuration to that of the conventional T cell receptor and the pre-T cell receptor: a disulfide-linked heterodimer non-covalently associated with the signal transducing CD3 subunits.

It should be noted that all of the CD3-associated proteins identified to date are antigenically and biochemically distinct, as indicated in Table I, Section 5.2, supra. Thus, p33 differs from all known CD3-associated proteins, being characterized by a MW of 33 kD, and an isoelectric point of 4.5, and co-precipitating exclusively with CD3∈ and TCRβ monoclonal antibodies. Although closest in size to the pTα chain, p33 is distinguished by isoelectric point and immunologically, i.e., in its failure to precipitate with pTα antiserum. p33 is thus thought to represent a distinct protein that contributes to the formation of a novel CD3/TCRβ/p33 surface receptor complex expressed on the FC population, which protein and/or complex plays an important role in allogeneic stem cell engraftment and the induction of tolerance. While the p33 protein and its complex has been discussed herein as being expressed by FC cells, it may be that this novel protein and/or complex is also found on other cell populations that are phenotypically similar to FC, such as dendritic or certain NK (Natural Killer) cells. For example, although classically associated with developing and mature T cells, CD3 expression has been documented in other cell types including NK cells, NKT cells and dendritic cells.

It has been hypothesized that facilitation of stem cell engraftment by the PC can be attributed to one of three potential mechanisms. The CD8⁺/CD3⁺ FC might act as an alloreactive effector cell at the time of transplantation, thereby lessening the recipient's ability to reject the donor stem cells. Alternatively, the FC might home to the BM and function by providing the engrafting stem cells with the appropriate signals and cytokines needed for self-renewal and differentiation. Lastly, the FC might assist in the re-education of the recipient immune system through an anergic or deletional mechanism, enabling the acceptance of donor stem cells.

The similarity of the FC phenotype to immature T cells, and of p33 to pTα, potentially favors a role for the FC as providing a deletion ligand for alloreactive cell types. Though the exact role of the FC population in the non-transplanted host is currently unknown, its ability to establish an environment of co-tolerance in the allogeneic setting (and its phenotypic similarity to developing thymocytes) supports the hypothesis that the FC may be a type of thymic precursor involved in the maintenance of self-tolerance.

The data presented herein demonstrate for the first time the expression of a novel p33 protein and/or TCRβ/p33 or CD3/TCRβ/p33 complex on the FC population that is associated with the facilitation of allogeneic hematopoietic stem cell engraftment and tolerance induction in vivo in the absence of acute GVHD.

We claim:

1. An isolated glycoprotein having: (i) a molecular weight of about 33 kD as determined by SDS-PAGE, (ii) a pI of about 4.5 as determined by IEF, (iii) the ability to form an association with the CD3 antigen or the TCRβ chain; and (iv) the ability to enhance hematopoeitic donor stem cell engraftment or induce donor-specific immunologic tolerance in transplant patients.

2. An isolated protein complex comprising the TCRβ chain in association with a 33 kD glycoprotein having a pI of about 4.5 as determined by IEF.

3. The isolated protein complex of claim 2 further comprising the CD3 antigen in association with the TCRβ chain and the 33 kD glycoprotein having a pI of about 4.5 as determined by IEF.

4. The protein complex of claim 2 or 3 wherein the TCRβ chain is linked to the 33 kD glycoprotein via disulfide bonding.

5. The protein complex of claim 2 or 3, which complex further has the ability to enhance hematopoeitic donor stem cell engraftment or induce donor-specific immunologic tolerance in transplant patients.

6. A method for enhancing hematopoeitic stem cell engraftment in vivo by administering to a patient in need thereof a therapeutically effective amount of a stem cell engraftment-enhancing protein having: (i) a molecular weight of about 33 kD as determined by SDS-PAGE, (ii) a pI of about 4.5 as determined by IEF, and (iii) the ability to form an association with the CD3 antigen or the TCRβ chain, in a pharmaceutically acceptable carrier.

7. The method of claim 6, wherein the engraftment-enhancing protein is in association with the TCRβ chain.

8. The method of claim 7, wherein the engraftment-enhancing protein and the TCRβ chain are further associated with the CD3 antigen.

9. A method for inducing immunologic tolerance in vivo by administering to a patient in need thereof a therapeutically effective amount of an engraftment-enhancing protein having: (i) a molecular weight of about 33 kD as determined by SDS-PAGE, (ii) a pI of about 4.5 as determined by IEF, and (iii) the ability to form an association with the CD3 antigen or the TCRβ chain.

10. The method of claim 9, wherein the engraftment-enhancing protein is in association with the TCRβ chain.

11. The method of claim 10, wherein the engraftment-enhancing protein and the TCRβ chain are further associated with the CD3 antigen.

12. A recombinant p33 protein.

13. A recombinant TCRβ/p33 protein complex.

14. A recombinant CD3/TCRβ/p33 protein complex.

15. A genetically-engineered host cell comprising a nucleic acid encoding a p33 protein or peptide, which host is capable of expressing a recombinant p33 protein or peptide.

16. The genetically-engineered host cell of claim 15 which further comprises a nucleic acid encoding TCRβ, which host is capable of expressing a recombinant TCRβ/p33 protein complex.

17. The genetically-engineered host cell of claim 16 which further comprises a nucleic acid encoding the CD3 antigen, which host is capable of expressing a recombinant CD3/TCRβ/p33 protein complex.
